(19) United States
(12) Patent Application Publication
(10) Pub. No.: US 2018/0154035 A1
(43) Pub. Date: Jun. 7, 2018

(54) DEVICE FOR CONTROLLED RELEASE OF FLUID

(71) Applicant: Microlin, LLC, Salt Lake City, UT (US)

(72) Inventors: Ashok V. JOSHI, Salt Lake City, UT (US); Aditya S. DESAI, Salt Lake City, UT (US)

(73) Assignee: Microlin ,LLC

(21) Appl. No.: 15/575,351

(22) PCT Filed: May 21, 2016
(86) PCT No.: PCT/US2016/033677

§ 371 Date: Nov. 18, 2017

Related U.S. Application Data
(60) Provisional application No. 62/164,650, filed on May 21, 2015.

(51) Int. Cl. A61L 9/12; A61L 9/04

(52) U.S. Cl. CPC A61L 9/127; A61L 9/042; A61L 2209/11; A61L 2209/131; A61L 9/125

(57) ABSTRACT

Delivery devices 100 for controlled discharge of a volatile fluid 118 at a relatively constant rate over a sustained period of time. Volatile fluid 118 may be dispensed in liquid or vapor phase. Rate of fluid discharge from a storage container 103 is controlled by a restriction element 124. Fluid 118 may be urged toward discharge by gravity, wicking, capillary activity, diffusion, pressure internal to the container, evaporation, and/or other fluid transmission through the restriction element 124.

### PRIORITY CLAIM

This application claims the benefit of the filing date of U.S. Provisional Patent Application Ser. No. 62/164,650, filed May 21, 2015, for DEVICE FOR CONTROLLED RELEASE OF FLIUID, the entire content of which is incorporated by this reference as though set forth herein in its entirety.

### TECHNICAL FIELD

This invention relates to devices for releasing fluid at a controlled rate over an extended period of time. Exemplary embodiments release volatile fluid for use in air freshening.

### BACKGROUND

It is often desirable to dispense one or more volatile fluid into a local environment at a relatively constant rate and over a sustained period of time. For example, it is desirable to dispense air freshener in certain bathrooms, or in the interior of an automobile. Preferably, an air freshener dispenser can operate to maintain the scent in the local atmosphere at a humanly perceptible and pleasing level, without need for constant human intervention. A similar device could be used to dispense, for example, mosquito repellant. It would be an improvement to provide a fluid-dispensing apparatus that can dispense a volatile fluid into an environment at a greater rate, a steadier rate, and/or over a longer sustained period of time, compared to commercially available devices.

### DISCLOSURE OF THE INVENTION

The invention may be embodied to provide an apparatus or device for dispensing volatile fluid in gas or vapor phase into a local environment. An exemplary such embodiment includes a container structured to hold the volatile fluid in liquid phase. A quantity of liquid volatile fluid is initially disposed inside the container, and is discharged through a first port. Desirably, the discharge rate of fluid is controlled to permit sustained and slow fluid discharge over a sustained period of time at a substantially constant rate. A preferred period of time is in excess of a week; desirably two weeks, three weeks, or more.

Rate of fluid discharge from a container may be controlled by a restriction element. One operable restriction element may include a first aperture associated with the first port. One such aperture may have a characteristic size and/or configuration effective to provide a desired fluid flow rate there-though. Sometimes, fluid flow rate may be controlled by structure associated with the aperture. For one non-limiting example, a restriction element may be formed by a porous membrane structured to provide a path for diffusion of the fluid from the inside to outside of the container. Membrane parameters, such as exposed and/or wetted area, thickness, and material of composition, etc., may be configured to produce a desired discharge of volatile fluid in vapor phase.

An operable restriction element can be embodied as a valve. A workable valve may even be configured to change in size over time, or due to change in temperature, to change a flow rate from the container. One workable restriction element is formed by a plug with at least one groove, the plug being structured to engage with the container such that the groove forms a discharge channel for the fluid. In some cases, a plug may include a plurality of grooves to provide a plurality of fluid flow paths from the container. Grooves may be substantially straight, or may have alternative shapes. For one example, a plug may provide one or more spiral fluid channel.

One workable restriction element includes a first aperture and a second aperture structured inter-cooperatively to produce a desired flow rate through one of the first or second aperture. Sometimes, a container may be inverted, or rotated, to cause fluid discharge from a different port or aperture. Such container re-orientation may provide for fluid discharge at a different rate through the other aperture.

Typically, a seal element is provided to resist undesired fluid motion (liquid/vapor discharge or air vent) through a port, or other path of potential fluid escape from the container, prior to placing the device into service. Desirably, the seal is removable in a tool-free operation. An exemplary seal element includes a foil membrane positioned to block a fluid discharge path from a container. Other workable seal elements non-exclusively include screw-off or snap-off caps, corks, and the like.

Fluid dispensing devices typically include some sort of anti-vacuum structure configured to resist a decrease in pressure inside the container, below the local pressure outside the container, due to discharge of the fluid. An anti-vacuum structure can include a second port that functions as an air vent that admits air into the container to compensate for a volume of discharged fluid. The air vent may be structured as a brake element to control fluid flow. In that case, the air vent may operate in harmony with a fluid discharge aperture to produce a desired fluid discharge rate. An anti-vacuum structure can also, or alternatively, include collapsible structure, such as a collapsible bag or portion of a container's wall. Another anti-vacuum arrangement within contemplation includes a gas-emitting element or compound that can even cause an increase in pressure inside the container compared to the local pressure outside of the container.

A fluid dispensing apparatus generally includes an emanator operably associated with the container and structured to dispense the fluid in gas phase into the atmosphere local to the container. Sometimes, an emanator is a separate element that is held in association with the container. Sometimes, and emanator may be, or include, the restriction element. It is within contemplation that an emanator may be formed by the container, itself. For one example, a porous membrane may operate as a restriction element and an emanator. A convenient emanator may be formed from an absorbent material made entirely or in part of woven fiber cloth, non-woven fiber cloth, woven synthetic cloth, non-woven synthetic cloth, natural fiber cloth, sponge or sponge-like materials, pads, blankets, membranes, and the like.

One embodiment provides an emanator that carries, or is associated with, a piercing mechanism configured to pierce the first seal when the container is placed in operable position to dispense fluid. Certain embodiments may include one or more charge reservoir associated with the container and structured to dispense an initial bolus or dollop quantity of fluid to the emanator when the apparatus is first placed into service to dispense volatile fluid.

Certain embodiments include a restriction element operable as a multi-function discharge valve structured to permit a step increase in instantaneous rate of fluid discharge from the container. For example, it is within contemplation that turning a container over, subsequent to a first period of time of use of the device, may permit fluid to discharge through a larger discharge aperture. Such a device can compensate for reduced head pressure due to a smaller reservoir of fluid remaining inside the container. In that case, the discharge valve is structured to permit manual operation to cause a step increase in discharge of volatile fluid by orientating the container to discharge volatile fluid from a different aperture.

In other embodiments, a discharge valve can be structured to cause automatic operation after a period of time in which the container has been in service to dispense the volatile fluid. For non-limiting example, a discharge valve may include a first open discharge aperture and a second discharge aperture initially obstructed to fluid flow by a plug material structured and arranged to degrade in the presence of volatile fluid contact. Operable plug material may be selected from polystyrene, polyethylene glycols, rubbers, polystyrene composites, glues, polyester, or other polymer composite which degrades in the presence of the volatile fluid. A discharge valve may also operate based upon a temperature in which the device is operating, or by way of some other inherent material characteristic. For example, a coefficient of thermal expansion of a valve element may be employed to cause an automatic opening or closing effect based upon temperature changes.

The invention may be embodied as an apparatus structured to dispense volatile fluid in gas phase into a local environment. A container holds a quantity of the volatile fluid in liquid phase. A first port is configured to permit discharge of the volatile fluid from the container. This embodiment also includes a restriction element structured to provide a controlled rate of discharge of the fluid at a substantially constant rate from the container over a period of time in excess of one week. The restriction element generally is associated with a first aperture and with the first port. A first seal operable to resist undesired fluid motion through the first port may also be included in certain embodiments. Desirably, anti-vacuum structure is included to resist a decrease in pressure inside the container, below the local pressure outside the container, due to discharge of the fluid. An emanator is operably associated with the container and structured to dispense the fluid in gas phase into the atmosphere local to the container.

The invention may be embodied as a container structured to hold a quantity of volatile fluid in liquid phase to permit escape of the fluid in vapor phase, or sometimes in liquid phase, into the environment local to the container over an extended period of time. A quantity of the fluid is initially disposed inside the container. A restriction element is provided to control the rate of discharge of the fluid from the container. A currently preferred restriction element includes a micro-molecular self-healing membrane structured to provide a controlled rate of discharge of the fluid from the container at a substantially constant rate over a period of time in excess of one week. A workable restriction element may include a polymer-based heat-shrink material or wet cell battery separator material. A preferred restriction element includes a porous membrane structured from polyolefin, polypropylene, or polyvinylchloride material, styrene-based polymer or rubber, and the like. A workable membrane confines volatile fluid in liquid phase, but permits fluid vapors to escape from the container.

An exemplary container may include a pouch, tube, or volume-defining enclosure formed at least in part from material through which the fluid may diffuse and which is desirably heat-sealable to define a volume in which the fluid is confined. An alternative workable container includes a rigid enclosure having a discharge opening blocked by the restriction element. In certain embodiments, the discharge opening can be disposed to permit fluid contained inside the container to wet the restriction element under influence of gravity.

Embodiments may also include a gas-generating compound disposed inside the container and operable in the presence of moisture to generate a gas effective to increase pressure inside the container over the local atmospheric pressure outside the container. Certain embodiments may include a volume-occupying structure disposed inside the container and structured to maintain a minimum volume of the container as fluid is permitted to escape from the container. One currently preferred volume occupying structure includes a cellulosic sponge, or other comparable material, arranged to wick fluid to maintain fluid contact with the restriction element over the effective life of the apparatus.

Embodiments may be used in conjunction with a support structure configured and arranged to hold a plurality of containers to increase a quantity of fluid in vapor form that may be discharged into the local atmosphere in accordance with the number of the containers, the support structure being arranged to permit circulation of local atmosphere around the containers held therein.

### BRIEF DESCRIPTION OF THE DRAWINGS

In the drawings, which illustrate what are currently regarded as the best modes for carrying out the invention and in which like reference numerals refer to like parts in different views or embodiments:

FIG. 1 is a is a view in elevation and in cross-section of certain components of a device structured according to certain principles of the instant invention; and

FIG. 2 is a view in elevation and in cross-section of certain components of an alternative embodiment structured according to certain aspects of the instant invention;

FIGS. 3A and 3B are views in elevation and in cross-section of certain components of another alternative embodiment structured according to certain aspects of the instant invention;

FIGS. 4A-E are views in elevation of certain components of another embodiment illustrating various stages of deployment;

FIG. 5 is a perspective view of another embodiment of a container;

FIGS. 6A-C are a top view, close-up of a portion of the top view, and a view in perspective, respectively, of a workable arrangement for a restriction element;

FIG. 7 is an X-Y plot illustrating fluid discharge data collected in an experiment using containers similar to that illustrated in FIG. 5;

FIG. 8 is a view in perspective of another embodiment;

FIGS. 9A and 9B illustrate views in elevation of an alternative gravity-operated embodiment that automatically changes a size of the total discharge opening after passage of time;

FIG. 10A is a cross-section side view in elevation taken along line 10A-10A in the embodiment in FIG. 10B;

FIG. 10B is a plan view in elevation if a pouch embodiment, FIG. 10C being an end view there-of;

FIG. 10D is an end view of an alternative cylindrical embodiment similar to that illustrated in FIGS. 10A-C;

FIG. 11 is a plan view of another pouch embodiment;

FIG. 12 is a view in elevation of a holding device for a plurality of pouches;

FIG. 13 is a cross-section view in elevation of an alternative embodiment;

FIGS. 14A-d are top, cross-section in elevation, front, and bottom views of another embodiment, respectively;

FIG. 15 is an X-Y plot of representative data collected by testing embodiments structured similarly to that illustrated in FIG. 13;

FIGS. 16A and 16B are plan and cross-section side views in elevation, respectively, of an alternative pouch embodiment;

FIG. 17A is a cross-section view in elevation of another embodiment;

FIGS. 17B and 17C are close-up views of a portion of FIG. 17A, with stopper structure being disposed at two alternative positions;

FIGS. 18A and 18B are plan and cross-section end views, respectively of another embodiment;

FIG. 19 is an end view of a solid puck embodiment;

FIGS. 20A and 20B are plan and end views of another embodiment; and

FIG. 21 is a cross-section side view of another embodiment.

### MODES FOR CARRYING OUT THE INVENTION

FIG. 1 illustrates certain components of an embodiment structured according to certain principles of the invention. The illustrated embodiment 100 includes a container 103 having a fluid discharge port 106, 106′ disposed at each of bottom end 109 and top end 112, respectively. Containers can be configured in any size and shape that may be desired, non-exclusively including cylindrical, spherical, brick, pancake or pill, and the like. Operable containers 103 may be rigid or flexible. An exemplary rigid container may be formed from a glass bottle, or stiff plastic walled enclosure. An exemplary flexible container may be formed from a thin-walled plastic bag, and the like. Sometimes, a container may stand on its own. Other times, a container may be supported by a housing or other skeletal structure. Operable materials of construction of a container include plastic, glass, metals, and the like, which can be formed into a desired shape and resist degradation by the confined fluid.

A device according to certain principles of the invention may sometimes include a housing (not illustrated in FIG. 1) in which to hold a container 103 in workable association with an emanator 115. The housing may be configured to provide a pleasing and unobtrusive appearance.

During manufacture of the device 100, a quantity of fluid 118 is loaded into, and initially confined in, a storage volume 119 defined by the container 103. Fluids 118 may encompass any fluid for which a controlled dispensing rate under the influence of gravity is desired. However, it is currently preferred to use the device 100 for dispensing a scented, or scent-carrying, fluid operable to release a fragrance into the local atmosphere. Accordingly, commonly dispensed fluids 118 are at least somewhat volatile in nature, and evaporate from an emanator 115 to disperse fragrance in the local area.

A common use of the device 100 is dispensing scent or fragrance into a room of a dwelling over a significant period of time, such as over a few weeks, a month, or more. The device 100 permits a slow discharge of fluid from the container 103 to impact onto the scent emanator 115. In general, the fluid 118 is dispensed from the container 103 under influence of gravity onto an emanator 115 in a drop-wise fashion. An operable emanator 115 includes paper, fiber mats, sponge materials, and other materials that permit fluid that is applied to one side to spread out and evaporate from the other side. Desirably, the emanator 115 is structured to resist leaking of fluid 118 from the device 100.

Removable seal elements 121 are typically placed over the first discharge port 106 and second fluid discharge port 106′, to resist leaking of fluid 118 during storage and transport. A consumer can remove the seals 121 when the device 100 is desired to be used. Operable fluid seal elements 121 non-exclusively include any conventional fluid sealing structure, including the illustrated foil wrappers that are adhered over the openings of the first and second fluid discharge ports. Alternative seal elements 121 within consideration include various caps, corks, screw-on and -off elements, and the like, which are well known to designers of fluid containers.

A restriction element, generally 124, functions to control rate of discharge of fluid 118 from confinement inside a container 103. A restriction element 124 may also, or alternatively, control the flow of a make-up gas into a container 103. An exemplary restriction element 124 may include a blocking element or plug 125 effective to resist liquid fluid flow through a discharge port, except through one or more aperture. Another exemplary restriction element 124 may permit controlled transmission of fluid 118 in a vapor phase from a storage container 103 that holds a quantity of liquid fluid 118. One such restriction element 124 may be formed from a section of porous membrane, such as polyolefin or polypropylene heat shrink material, or battery separator material operable to separate the cathode and anode in a wet cell battery.

In FIG. 1, restriction element 124 includes top and bottom plugs 125, 125′, which are disposed to block uncontrolled fluid flow through the fluid discharge ports 106, 106′, respectively. A restriction element 124 can be integral with the container 103, or may be a separate component that is affixed in place. As illustrated in FIG. 1, restriction element 124 includes a fluid-impermeable blocking disk or plug 125 (e.g. plastic), that can be affixed in place by conventional manufacturing techniques, including adhesive, solvent, interference fit, heat and friction welding, and the like. Discharge aperture 127 permits fluid flow through disk 125. Another operable restriction element 124 may be formed, for example, by drilling a suitably small hole, or discharge aperture 127, through a wall of a container 103.

Flow rate produced by a container 103 is a function influenced by several elements, including depth of fluid, aperture length, aperture characteristic size such as diameter (or cross-section area), fluid viscosity and other fluid properties, and others. In certain embodiments, fluid flow rate is produced from gravity effect on fluid in the container 103, only. That is, no additional pressure source, or propellant is required to produce the desired flow rate in certain embodiments. In other embodiments, a pressure-producing element may be included to urge fluid flow at a desired rate over an extended period of time. In other embodiments, a portion of a container 103 may function as both an emanator and a restriction element 124, and permit fluid egress from a container 103 in a vapor state at a desired controlled rate.

With continued reference to FIG. 1, a first aperture 127 is disposed to permit fluid 118 pass through restriction element 124 at the first discharge port 106. A second aperture 130 functions as a vent disposed to permit entrance of atmosphere into the container 103. Apertures 127, 130 can inter-cooperate, with fluid flow through a discharge aperture 127 being retarded by a vent aperture 130 sized to admit air into the container at a sufficiently low rate as to act as a brake, and reduce flow through the discharge aperture 127.

Structure can be provided to compensate for the dispensed volume of fluid 118 and permit sustained release of the fluid 118. Sometimes, air vent structure, such as aperture 130, may be provided to directly provide a make-up volume of gas inside the container as fluid 118 is released. Illustrated apertures 127,130 are discernable through-holes, and are distinguished over a pore in a membrane. However, certain embodiments according to the invention may have a single discernable discharge aperture 127, and a vent (“aperture”) may be formed by alternative gas-passing structure effective to admit air into the container, as required. Other embodiments may be structured to alter the shape of the container and thereby permit continued release of fluid. Other embodiments may include off-gassing elements inside the container to provide make-up gas volume. In the latter case, the off-gassing elements may even provide pressure to urge flow of fluid 118 through an aperture 127 at a desired rate.

As a non-limiting example, it is also within contemplation that one or more discharge aperture 127 may be included in a collapsible container 103 to avoid need for a vent aperture 130 to admit exchange atmosphere to replace a volume 133 of dispensed fluid 118 as fluid level 136 drops. Also, it is within contemplation that certain embodiments may be structured to permit sustained release of volatile fluid (e.g., vapor) directly through a wall element of a container 103. In that case, one suitable wall element may include a porous membrane. Also in that case, the fluid 118 in a liquid state is confined inside the container 103, but the wall element may permit transit of vaporized fluid 118 to the local atmosphere outside of the container. The wall element of such a container 103 may function as an emanator 115.

Apertures 127, 130 sized to produce the desired fluid flow rates are fairly small, and can be produced by laser drilling, needle poking, and other conventional manufacturing techniques. For convenience, an aperture 127, 130 may be characterized in this disclosure as having a diameter for a characteristic size. However a workable aperture may have some other cross-section than circular, and some other corresponding characteristic size designation.

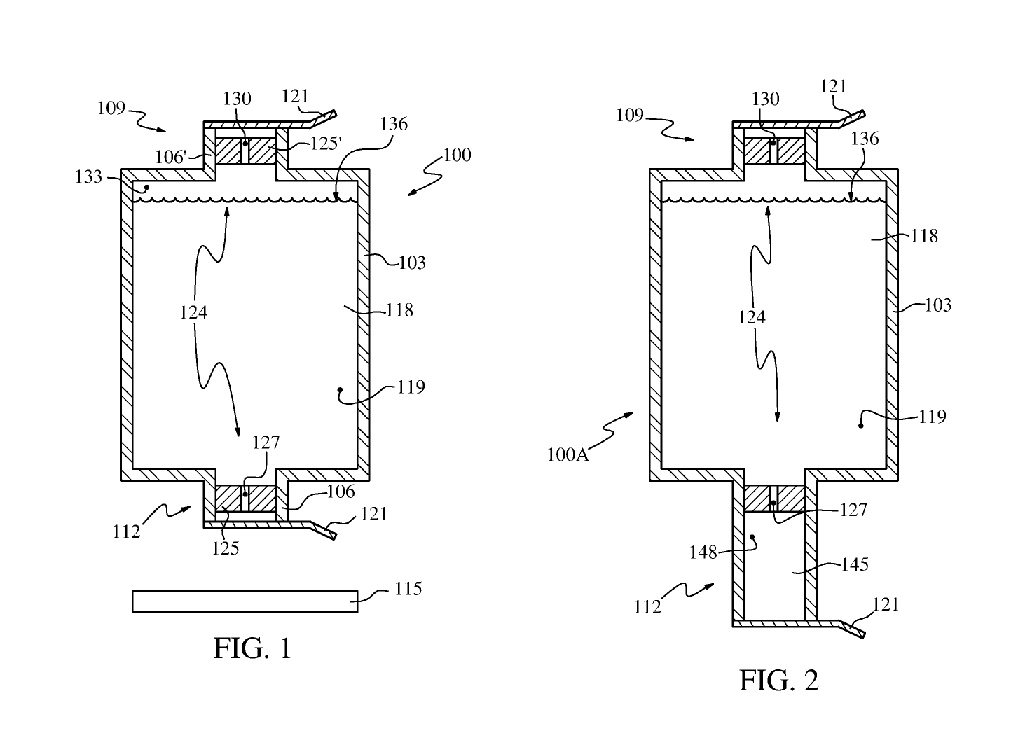

As illustrated in FIG. 2, a second workable embodiment 100A may include a container 103 structured to include a charge reservoir 145 in which to hold an initial charge volume 148 of fluid with which to saturate an emanator (not illustrated) upon first deployment of a device 100A. When present, the initial fluid charge volume 148 is desirably sized to dispense a sufficient amount of fluid 118 as an initiating charge onto an emanator to produce a substantially immediate burst of fragrance in the room at a desired level of perceptibility. The embodiment 100A then provides sufficient fluid flow to maintain the fragrance at an operable level.

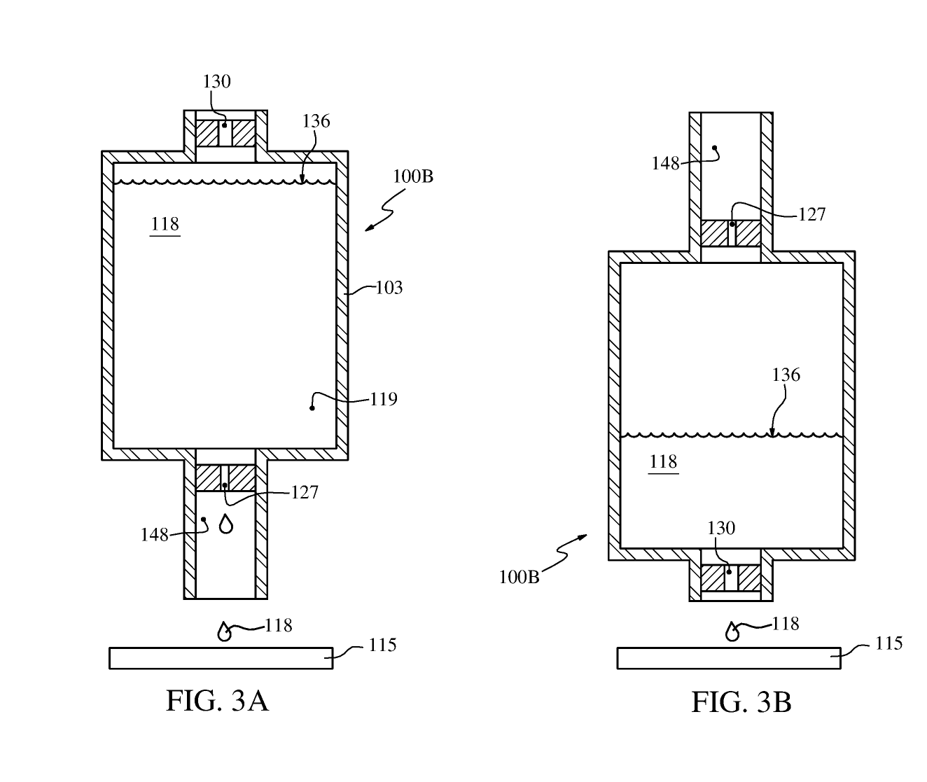

As illustrated in FIGS. 3A and 3B, a container 103 may be structured to provide a plurality of flow rates. The embodiment 100B in FIG. 3A and 3B has apertures of two sizes, which produce two respective flow rates under equivalent conditions. The aperture sizes are desirably sized to compensate for reduction in fluid pressure (head loss) due to dispensing part of the fluid. That is, the larger aperture 130 can be sized to increase the flow rate over the flow rate from the smaller aperture 127 when the fluid level 136 has dropped by say, half way. Flow rates between different apertures may vary by perhaps 50%, or less, to 500%, or more. Desirably, the flow rate from an inverted half-full container 103 is similar to the flow rate from a full and right-side-up container 103. In that case, fragrance may be dispensed at a more consistent and uniform level over the life of a dispensing device, such as 100B.

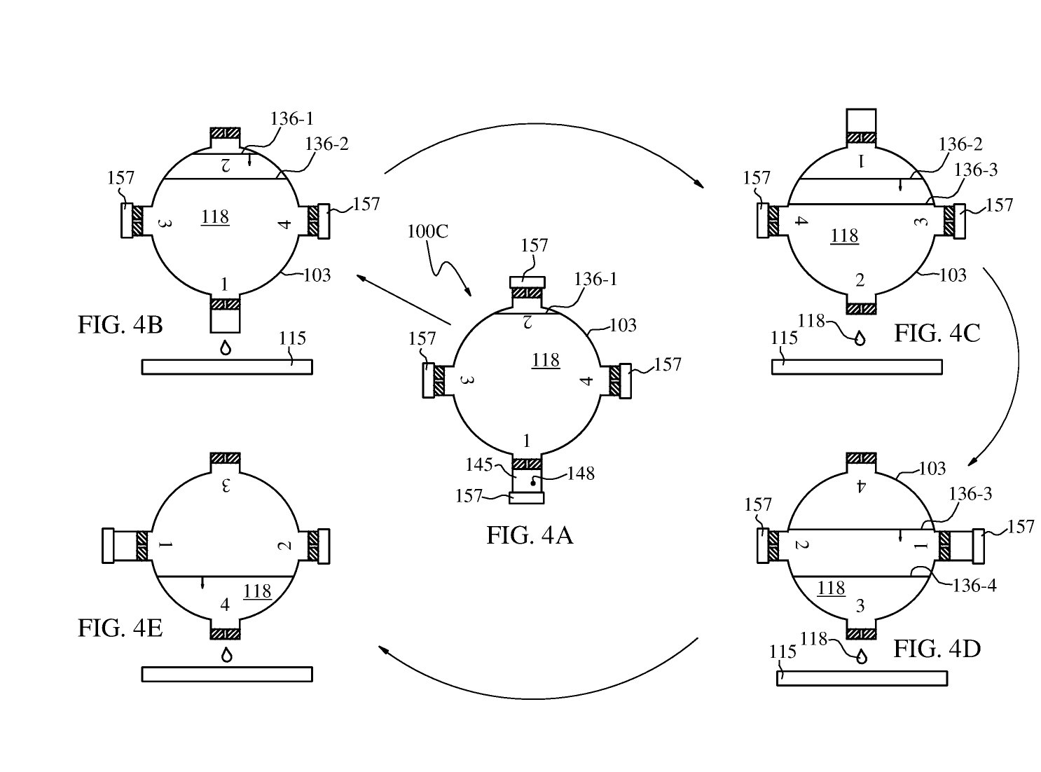

FIGS. 4A-E illustrate an extension of the principle illustrated in FIGS. 3A and 3B. FIG. 4a represents a dispensing device, generally indicated at 100C, in condition as-sold to a consumer. Fluid-sealing caps 157 are removed from discharge ports 1 and 2, and the container 103 is associated with an emanator 115 of a fragrance-dispensing device (FIG. 4B). In the case (as illustrated) where a charge reservoir 145 is included in the container 103, an optional burst of fragrance may be deployed by pouring a fluid charge volume 148 onto the emanator 115. The container 103 is then deployed such that fluid 118 is dispensed onto the emanator 115 from port 1 at a first or initial discharge rate. The fluid discharge rate changes slightly over time due to head loss as fluid level drops from initial level 136-1 to level 136-2, and potentially other factors.

After a first period of time (e.g., a week), the container 103 is everted to begin dispensing fluid from port 2 at approximately the same rate as the initial rate (FIG. 4C) during a second period of time (e.g., a week). The discharge aperture of port 2 is sized larger than the discharge aperture of port 1 to cause the desired flow rate under the conditions of operation during that time period. During the second period of time, level of fluid 118 drops from 136-2 to 136-3.

After the second period of time, the container 103 is then rotated by 90 degrees to dispense fluid from port 3 at approximately the same rate as the initial rate (FIG. 4C). The discharge aperture of port 3 is sized larger than the discharge aperture of port 2 to account for reduced head pressure available as fluid 118 drops from level 136-3 to 136-4. Prior to repositioning the container 103 for the third period of use, if fluid level 136-3 in the container 103 requires (and as illustrated), fluid sealing caps 157 may be exchanged from ports 3 and 4 to ports 1 and 2 (FIG. 4D).

After lapse of a third time increment, the container 103 may be rotated by 180 degrees to again restore the fluid flow rate to approximate the initial flow rate by discharge of fluid 118 through a largest-size discharge aperture disposed in port 4 (FIG. 4E). Container 103 may then be left in service until fluid 118 drops from level 136-4 sufficiently to reduce effectiveness of the device, or until container 103 is empty and emanator 115 may be dry.

### EXAMPLE

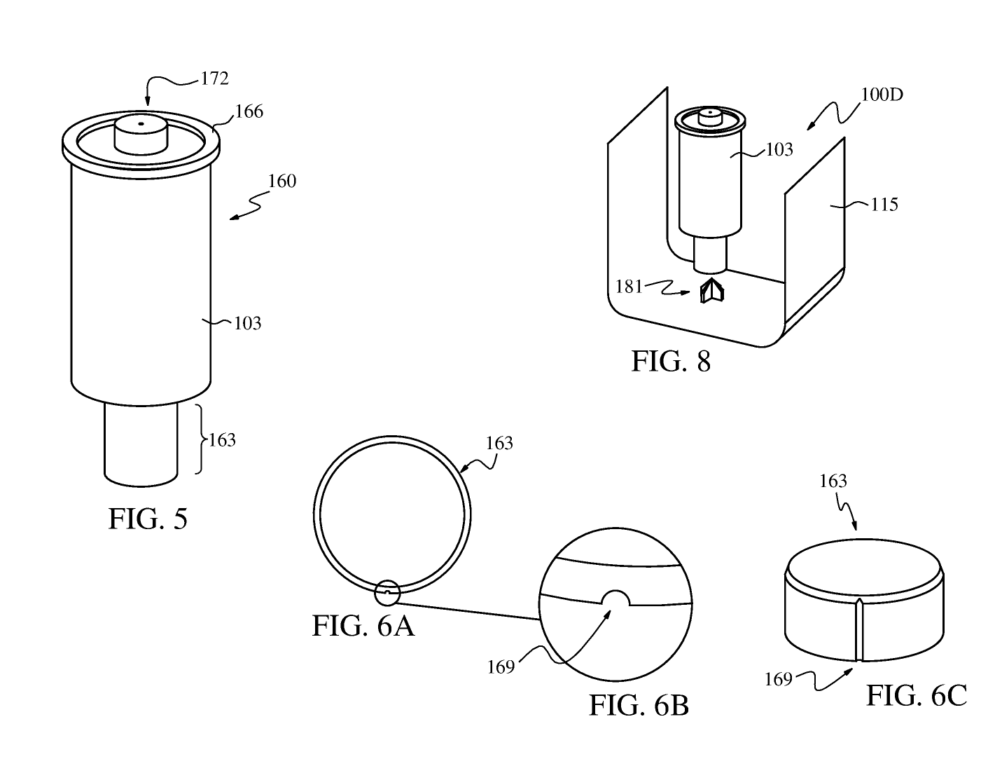

30 cc of vacuum oil was placed into each of two approximately cylindrical containers 103 of the type indicated at 160 in FIG. 5. The open-ended cylindrical container 103 in FIG. 5 is capped on each end. A restriction element, or valve for controlled fluid discharge, is disposed in a restrictor end cap 163 structured similarly to that illustrated in FIGS. 6A, 6B, and 6B. Container No. 1 had a plurality of discharge apertures at its bottom, and container No. 2 had a similarly disposed discharge apertures sized about 30% larger than container No. 1. The top of container 103 is substantially closed-off by top-end cover 166. Apertures 169 were formed as fluid transporting grooves extending the length of side-wall structure of the restrictor cap 163. The remainder of the cap 163 formed a fluid-blocking plug. The containers 160 were oriented so that the restriction caps 163 were disposed at the bottom of each container in the fluid discharge path. Each container 160 included a 2 mm diameter through-hole disposed in the top-end cover 166 and operating as an air vent 172. Fluid was allowed to flow under influence of gravity, and collected fluid was measured.

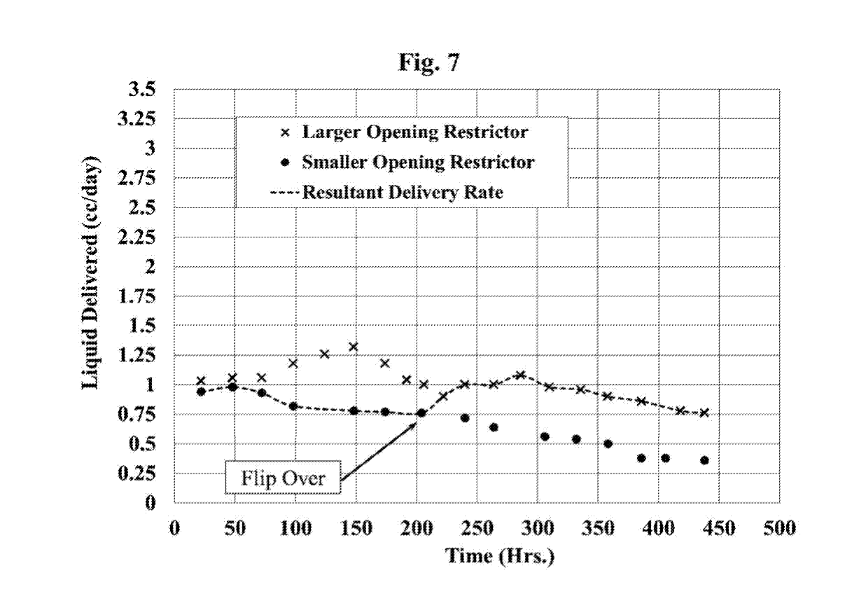

Experimental results are set forth in the X-Y plot illustrated in FIG. 7. After approximately 15 cc of fluid was discharged from each container, container No. 2 showed a higher rate of fluid discharge than container No. 1. Accordingly, it is predicted that a container having a top hole sized 30% larger than the discharge hole of container No. 1 may be turned upside-down to produce a fragrance discharge rate approximately as indicated in the plot of FIG. 7 by the dashed line. A step-change (pointed out by the “Flip over” note in FIG. 7) is imposed on the fluid delivery rate from the container at the time when the container is turned upside-down. On average, the discharge for such a double-ended container is closer to a desired consistent rate.

Vacuum oil was used in this experiment to correlate with results expected for fragrance oil. Vacuum oil is nonvolatile, and has a density consistent with fragrance oil. Fluid delivery measurement was easy and accurate.

Another embodiment of a fluid delivery device within the ambit of the invention is generally indicated at 100D in FIG. 8. A piercing mechanism, generally 181, may be disposed to form a fluid path through a seal (e.g., a foil seal 121, FIG. 1) to drain an initial charge of fluid onto an emanator 115 upon installation of a container 103 into a fragrance-dispensing device. Valve structure included in the mechanism 181, or restriction elements (such as 124 in FIG. 1), may then permit flow of a fluid 118 from container 103 at a desired rate over an extended period of time.

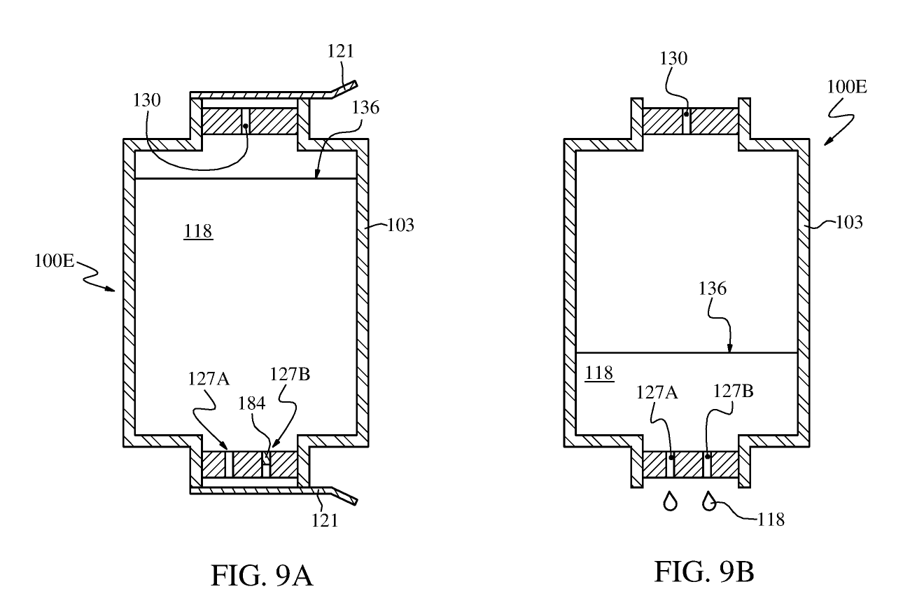

FIGS. 9A and 9B illustrate an embodiment 100E that automatically changes a size of the total discharge opening after passage of time. The fluid container includes a plurality of apertures, at least two of which (127A and 127B) are disposed to permit fluid discharge under the effect of gravity, and one of which is a vent 130 disposed to permit air to enter the container 103. Initially, discharge aperture 127B is occluded by a material that degrades in about 14 to 40 days. The occluding material 184 may be considered as discharge valve having an automatic “stopper” that automatically is removed after a period of time.

When placed into service, fluid 118 is free to discharge from the first aperture 127A, and the container discharges fluid 118 at a first approximately constant rate (e.g. +20%) as the fluid level 136 drops. As the fluid level 136 drops, the flow rate decreases until the occluding material 184 degrades, and permits the second opening 127 to release fluid. When the second opening 127 becomes active, the total discharge area is increased over the area provided by first aperture 127A. Consequently, the total fluid discharge rate increases to compensate for the reduced head pressure produced by the reduced depth of fluid 118. Therefore, fluid 118 may be automatically discharged by the device at a relatively constant rate over a longer period of time, such as a 30 to 60 day period.

An extension to this same principle can be effected by three or more discharge apertures 127, with additional discharge apertures 127 being freed to release fluid at various times. That is, the occluding “stoppers” 184 can be configured to degrade and permit fluid flow at different times. Stopper size and conformation can be design parameters. For example, a longer stopper disposed inside a lumen of a discharge aperture 127 will take longer to degrade, thereby providing more time before fluid can discharge through the associated aperture 127.

An automatic “stopper” 184, or time delay valve structure, may be made from any material that slowly degrades in the presence of a volatile fluid, such as a fluid fragrance. Workable stopper materials non-exclusively include: polystyrene, polyethylene glycols, rubbers, polystyrene composites, glues, polyester and other polymer composites which degrade in the presence of fluid fragrances, or other volatile fluids that may be used in certain embodiments.

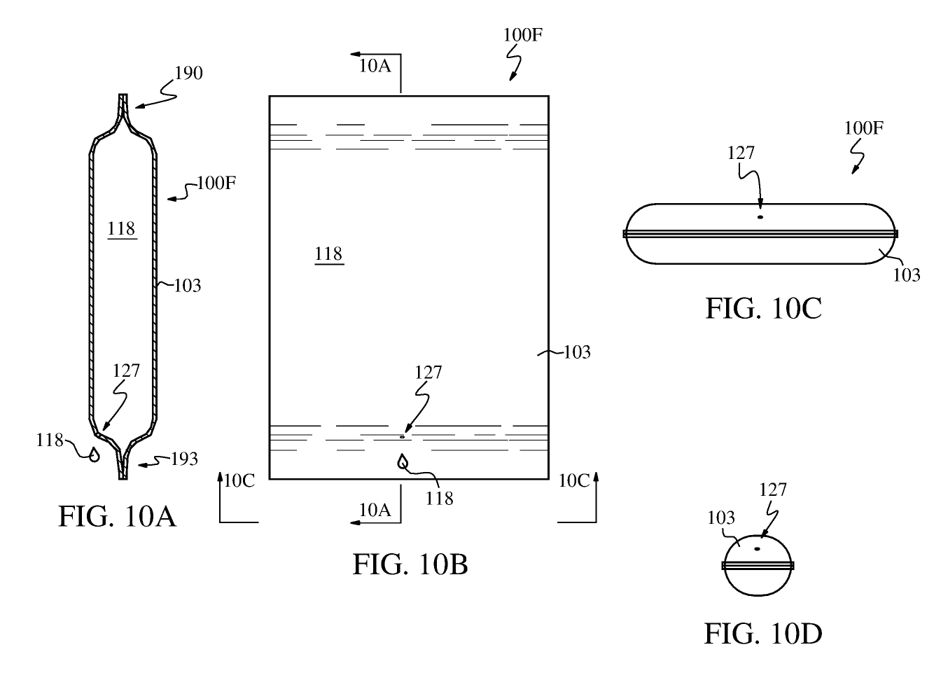

FIG. 10A illustrates a side view of an embodiment 100F that may be configured as a pouch or tube. As shown in FIGS. 10B and 10C, a pouch may be formed by sealing opposite ends of an elongate tube that is partially filled with volatile fluid 118. Preferably, first end 190 and second end 193 are heat-and-compression sealed. As shown in the end view of FIG. 10D, a more cylindrical embodiment may be formed by sealing ends of a substantially full tube. Alternative embodiments may be formed by, for example, folding a membrane, and sealing top and bottom plies together around the other three sides of a perimeter.

A flexible container 103 of the type illustrated in FIGS. 10A-D may be formed from relatively thin plastic, rubber, urethane, silicone, or other flexible and fluid-resistant compound or material. One preferred container 103 is made from a porous membrane, such as polyolefin or polypropylene heat shrink tubing, or material suitable for use as a divider in a wet cell battery. In such an embodiment, a discharge aperture 127 may, or may not, be included. Volatile fluid 118 may simply diffuse through the container's wall at a desired rate. In the case where container 103 is formed from a relatively impermeable material, discharge aperture 127 will be included, and will be structured to permit discharge of fluid 118 at a desired rate. A flexible container of the type illustrated in FIGS. 10A-D can collapse as fluid 118 is discharged, and thereby, resist forming a vacuum or internal pressure that is lower than the local pressure exterior to the container.

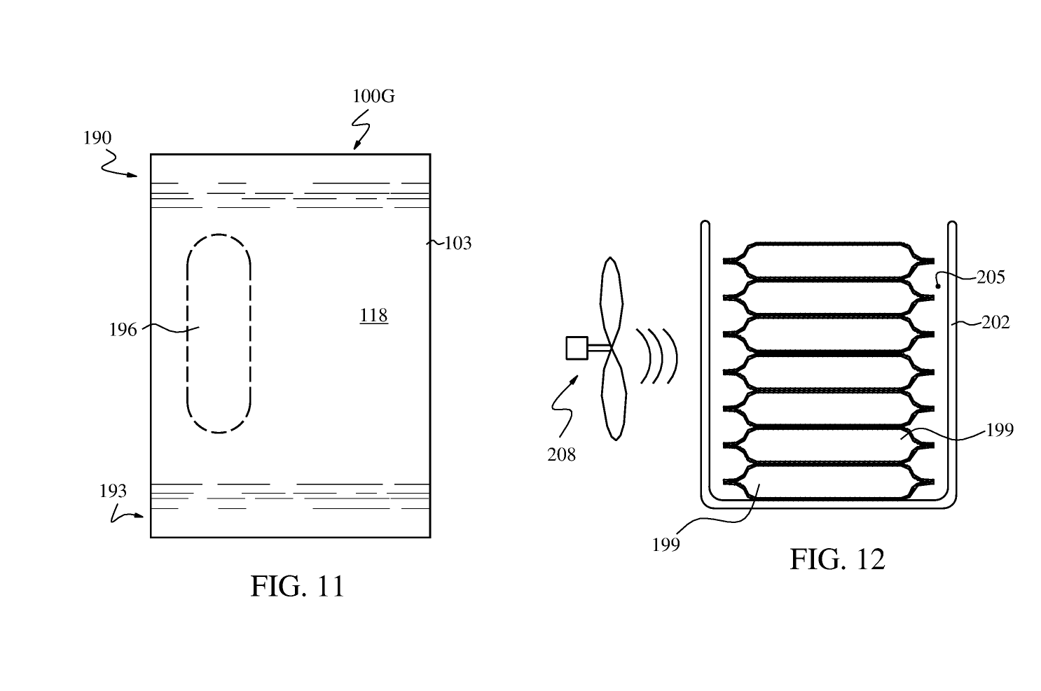

Embodiment 100G in FIG. 11 illustrates a flexible container 103 formed from a porous membrane tube, and sealed at opposite ends 190, 193. Volatile fluid 118 diffuses through the wall of the container 103 to treat the local area in which the embodiment 100G is deployed. In the embodiment 100G, substantially the entire container 103 operates as a restriction element 124 and also as an emanator 115.

Sometimes, an internal pressure-forming substance 196 is included to further urge transport of the fluid through the container wall. A workable substance 196 is a gas-generating chemical compound that is not activated by the volatile fluid 118, but that can be activated by moisture, or some other activation agent, at a desired time. A workable gas-generating substance 196 includes acetic acid and Sodium Carbonate. Moisture from humidity in the air at the site of deployment of embodiment 100G can permeate into the container 103 to activate the gas-generating element 196.

Individual embodiments may dispense volatile fluid at a characteristic rate. Sometimes, it is desirable to dispense fluid 118 at a higher rate than a single embodiment can provide. Therefore, provision may be made to combine embodiments to deploy a plurality of fluid emitters in a space to be treated (e.g., in a large bathroom). As one example, FIG. 12 illustrates a plurality of pouches 199 stacked in a cage 202. A pouch 199 may be structured similarly to embodiment 100F or 100G, for non-limiting example. The cage 202 simply provides structure to hold a plurality of pouches 199 in a volume 205 that permits circulation of air to dispense fluid 118 into the local environment.

Pouches 199 may be spaced apart by racks (not illustrated) to increase exposed emanator area. It is within contemplation to include a fan 208 to assist in treating the local atmosphere with fluid 118. It is further within contemplation that wall portions of a cage (not illustrated) may be arranged as valve elements to permit air circulation over a subset of pouches. In that way, a particular volatile fluid (e.g., a fragrance) may be selected for dispensing into the local atmosphere at one time, and a different volatile fluid may be selected for dispensing at another time.

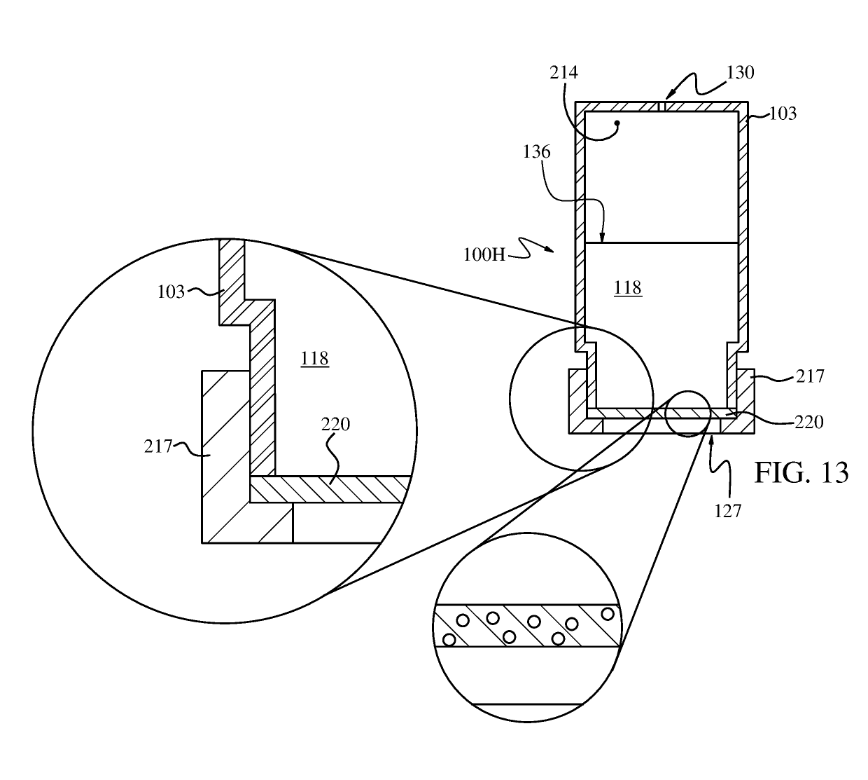

The embodiment 100H in FIG. 13 includes a rigid container 103 that defines a volume 214 in which to store volatile fluid 118. Cap 217 holds membrane 220 in operable association with container 103. Cap 217 provides a seal around a perimeter of membrane 220 and forms a port 106 through which fluid 118 may be discharged from the rigid container 103. One workable membrane 220 permits fluid 118 to diffuse through itself under the effect of gravity, or sometimes, when augmented by internal pressure in volume 214. Illustrated membrane 220 operates as a restriction element 124 that permits fluid 118 to diffuse. Another workable membrane may be impermeable to volatile fluid 118, in which case a discharge aperture or valve of some sort may be provided.

A currently preferred membrane 220 is formed from a sheet of SBR. An SBR membrane 220 can function as both a restriction element 124 and an emanator 115. Other workable materials include porous plastic-like and plastic materials, such as polyolefin, polypropylene, and polyvinylchloride heat shrink tubing, battery separator material, and the like. An air vent 130 permits fluid level 136 to drop without causing a vacuum inside container 103. In the illustrated vertical disposition of embodiment 100H, membrane 220 forms an emanator 115 having a wetted surface of constant size until substantially all fluid 118 is dispensed.

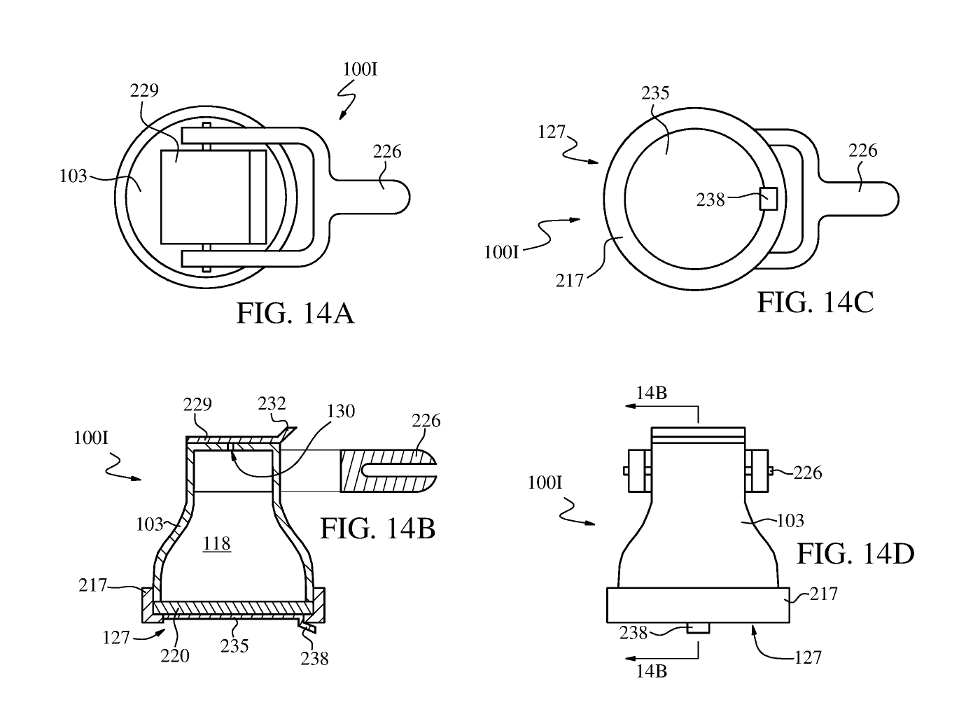

Embodiment 100I in FIGS. 14A-D is structured somewhat similar to embodiment 100H but is further adapted for use as an automobile air freshener. A clip 226 permits container 103 to pivot, and serves as an anchor to hold the container 103 in registration with an automobile's air vent. Illustrated top seal 229 is removable, and tear-off tab 232 is provided to assist in removal of seal 229. Similar seal 235 and tab 238 structures are shown on the bottom of the container 103. The seals 229, 235 are removed by the consumer prior to use of the air freshener 100I. Volatile fluid 118 is then diffused through membrane 220 and dispersed into the air stream from the vent.

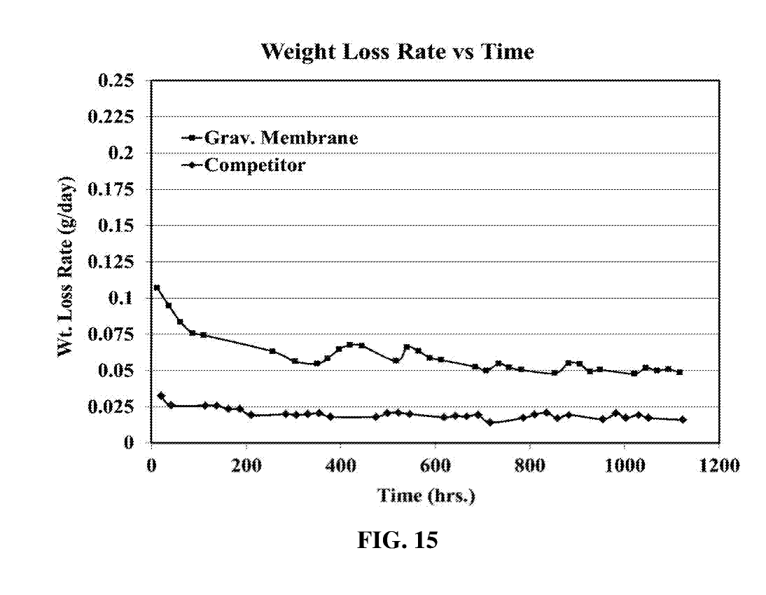

FIG. 15 illustrates averaged data collected from measuring output of two types of air fresher devices. The first type is structured similar to the embodiments in FIG. 13, and is characterized as a gravity membrane device. The second type is of a car air freshener that is commercially available under the trade name Febreze CAR vent clip and utilize a porous polymer membrane. A gravity membrane device includes a rigid body reservoir, which can be made out of a plastic or metal, and a micro molecular self-healing SBR membrane disposed at the bottom. The membrane thickness was 1/32″ and micro channels were pierced into it. The membranes were soaked in fragrance before being cut to size to fit into the device. Exposed membrane area was 1″ in diameter and 5 cc of fragrance was used. Air flow is expected to effect the delivery rates. However, the tests were conducted without any specialized air flow. It is expected that the delivery rates would be higher when used in combination with automobile air vents.

The fluid 118 may be inserted into the device before securing the membrane or may be injected into the device from an aperture on the top, which may or may not be closed once the fluid is injected. The fluid delivery is a result of molecular interaction between the fluid and the membrane material and/or membrane porosity and/or micro channels. Several fragrance delivery devices have been tested for about 45 days. The gravity membrane devices were sized to act as air fresheners for a small space such as the interior of an automobile. As seen in FIG. 15, the delivery rate obtained by the gravity membrane device was considerably higher than the commercially available product.

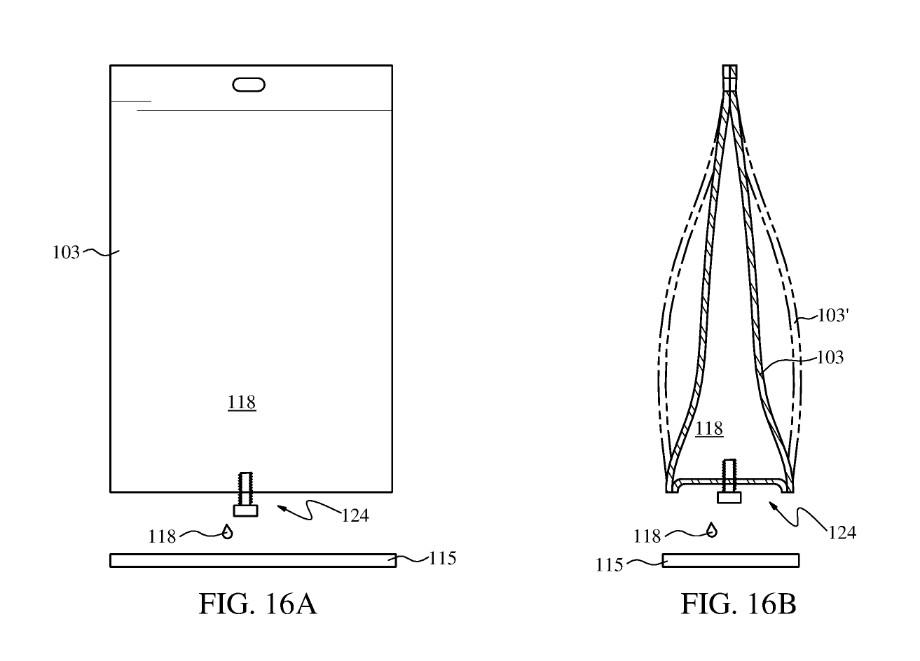

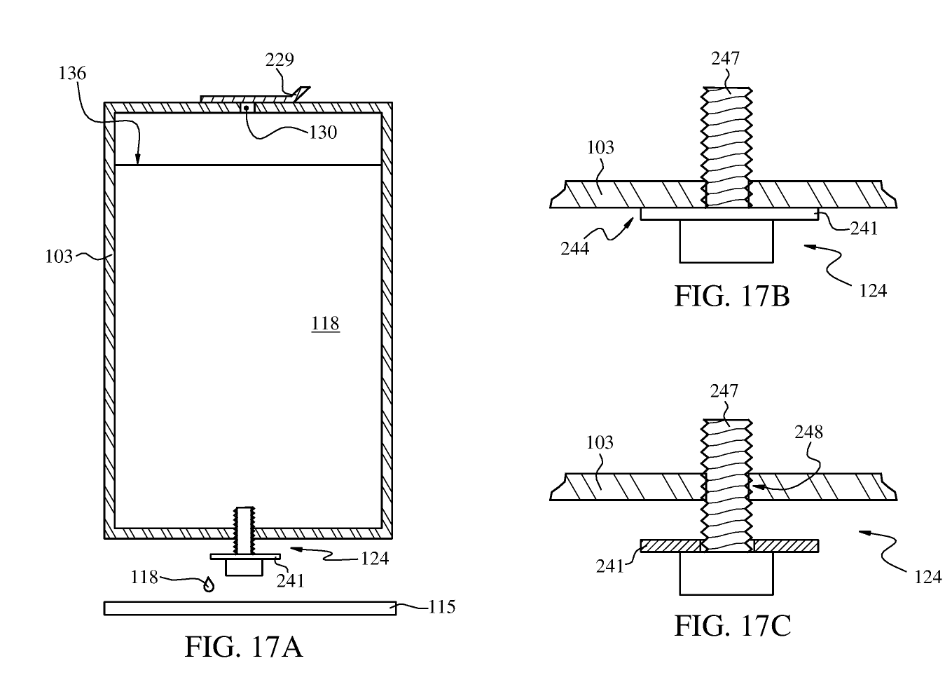

The collapsible walled embodiment illustrated in FIGS. 16A and 16B can be characterized as a plastic bag having a restriction element 124 disposed in penetration through its bottom wall. As fluid drains slowly onto the emanator 115, the walls collapse (e.g., moving from the position of phantom line structure 103′ to solid line structure 103). The rigid-walled embodiment illustrated in FIG. 17 includes an air vent 130, which is initially sealed against air and fluid passage by a removable seal 229. The air vent 130 in FIGS. 17 and collapsible walls in FIGS. 16 are exemplary structures that can resist formation of a vacuum inside a container 103 due to reduction in fluid level 136 during operation of the fluid-dispensing device.

As illustrated in FIGS. 16A-B and 17A-C, a restriction element 124 may be formed by a threaded fastener 247, such as a screw or bolt, which is threaded into a penetration hole of suitable size. The root of the thread, generally indicated at 248, forms a spiral channel to permit fluid flow from the container 103. This sort of a valve element is exemplary of an element that may be added to other embodiments, such as to the SBR membrane 220 in FIG. 13, in a mix-and-match operation.

With particular reference to FIGS. 17A-C, a resilient washer 241 may be included to facilitate forming a fluid-tight seal, generally indicated at 244. The coefficient of thermal expansion of a container 103 and a threaded fastener 247 may be selected to cooperate and deliver a desired effect on fluid discharge rate responsive to a temperature changes in the environment in which an embodiment of a fluid dispensing device is placed into service.

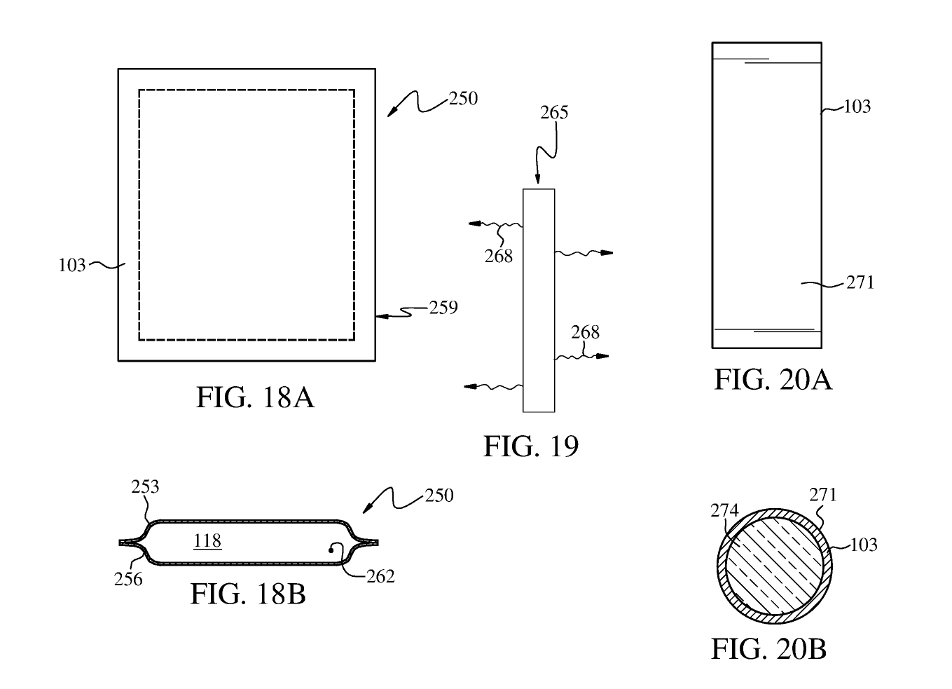

FIGS. 18A and 18B illustrate a patch, generally 250, that is formed by sealing top ply 253 and bottom ply 256 around a perimeter 259 of a compartment 262. Volatile fluid 118 is loaded into the compartment 259. Fluid 118 can be injected into compartment 259 by a syringe through a self-sealing passage formed in a ply by a needle. It is currently preferred that at least one of ply 253, 256 is made from SBR and functions as a restriction element 124 and an emanator 115. FIG. 19 illustrates an alternative off-gassing element that may be characterized as a puck, generally 265, which is made from a volume of SBR, and soaked for a period of time in volatile fluid. The puck 265 may then be placed into a room, and the volatile fluid is released by off-gassing from the puck over a period of time, as indicated by arrows 268.

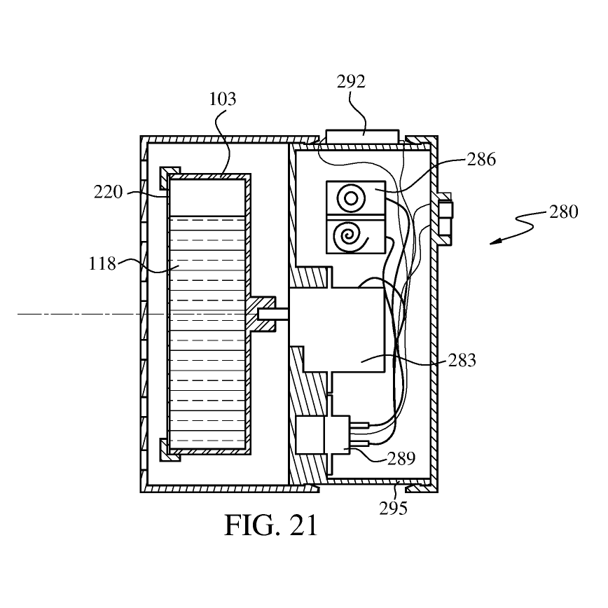

Variables that effect the amount of fluid released in vapor phase into the local atmosphere include total emanator surface area, and amount of emanator surface area that is wetted by fluid 118. FIGS. 20A and 20B illustrate an embodiment that is structured to maintain a substantially constant emanator size and wetted condition until the internal supply of fluid 118 is exhausted. Embodiments with similar capability are illustrated in FIGS. 13 and 21. The container 103 in FIG. 21A may be formed as a tube 271 from a porous plastic or plastic-like membrane (e.g. heat shrinkable polyolefin) and heat sealed at opposite ends. A sponge 274, or other moisture-wicking material, is placed inside the tube and saturated with fluid 118 prior to sealing the last end. Excess fluid 118 may also be added. The sponge 274 maintains the emanating surface of the tube 271 in a wetted state and size, thereby providing a long-term and consistent discharge of fluid 118 in vapor state into the local environment in which the tube 271 is placed into service. It is within contemplation to employ shredded SBR, or some other carrier that is saturated with volatile fluid, as an alternative to a sponge 274.

A self-powered fluid dispensing device, generally 280, is illustrated in FIG. 21. Embodiment 280 includes a motor 283, such as a DC servo motor, coupled to a container 103 in which fluid 118 is stored. Motor 283 is operable to rotate container 103 to maintain a large wetted area of membrane 220. A power source 286 is operably connected through a controller 289 to the motor 283. A workable controller may include a programmable logic controller (PLC), or even a simple on/off switch. As illustrated, an operable power source may include a battery or super capacitor that can be in-circuit with a recharging structure, such as solar panel 292. Other provisions for recharging or replacing the power source may be made in alternative embodiments. Panel 292 can conveniently be disposed on top of the housing or body 295 to receive solar radiation. The body 295 may carry structure adapted for attachment to a wall or car vent, for examples.

Membrane 220 may include a self-sealing port to permit filling container 103 with volatile fluid, avoid forming a vacuum inside container 103, and to resist leaking of the fluid 118 during conventional use. A workable membrane 220 may be structured from styrene-based polymer or rubber, with SBR being preferred. Certain membranes 220 may include micro-channel piercings, or other restriction elements 124, or valve or fluid channel elements, to promote delivery of fluid 118 to the local environment. Materials of construction of a body 295 may nonexclusively include polymers, such as Polypropylene, Polyethylene, Teflon, CPVC, and the like.

Although the invention has been described with regard to certain preferred embodiments, the scope of the claimed invention may be defined by the appended claims. However, any element or group of elements described with respect to any particular illustrated or discussed embodiment may be workably combined with any other element or group of elements of any other illustrated, described, or inherent embodiment in a mix-and-match operation to create a resulting embodiment within the ambit of the instant invention.

1.-20. (canceled)

21. An apparatus structured to dispense volatile fluid in gas phase into a local environment, the apparatus comprising:
a container structured to hold a quantity of the fluid in liquid phase;
a quantity of the fluid disposed inside the container; and
a restriction element comprising a micro-molecular self-healing membrane structured to provide a controlled rate of discharge of the fluid from the container at a substantially constant rate over a period of time in excess of one week, wherein:
the container comprises a pouch, tube, or volume-defining enclosure formed at least in part from material through which the fluid may diffuse and which is heat-sealable to define a volume in which the fluid is confined; and further comprising:
a volume-occupying structure disposed inside the container and structured to maintain a minimum volume in the container as fluid is permitted to escape from the container, the volume-occupying element being arranged to wick fluid to maintain fluid contact with the restriction element over the effective life of the apparatus.

22. (canceled)

23. The apparatus according to claim 21, further comprising:
a gas-generating compound disposed inside the container and operable in the presence of moisture to generate a gas effective to increase pressure inside the container over the local atmospheric pressure outside the container.

24. (canceled)

25. The apparatus according to claim 21, wherein:
the volume occupying structure comprises a cellulosic sponge.

26. The apparatus according to claim 21, wherein:
the container comprises a rigid enclosure having a discharge opening blocked by the restriction element, the discharge opening being disposed to permit fluid contained inside the container to wet the restriction element under influence of gravity.

27. The apparatus according to claim 21, further comprising:
a support structure configured and arranged to hold a plurality of containers to increase a quantity of fluid in vapor form that may be discharged into the local atmosphere in accordance with the number of the containers, the support structure being arranged to permit circulation of local atmosphere around the containers held therein.

28. The apparatus according to claim 21, wherein:
the restriction element comprises a polymer-based heat-shrink material or wet cell battery separator material.

29. The apparatus according to claim 28, wherein:
the restriction element comprises a membrane structured from SBR, polyolefin, polypropylene, polyvinylchloride, or other material capable of confining volatile fluid in liquid phase inside the container while permitting escape from the container of volatile fluid in vapor phase.

30. The apparatus according to claim 21, wherein:
the micro-molecular self-healing membrane comprises a styrene-based polymer or rubber compound.
